(19) United States
(12) Patent Application Publication
(10) Pub. No.: US 2017/0197017 A1
(43) Pub. Date: Jul. 13, 2017

(54) IMAGE-GUIDED IRRIGATING SUCTION CANNULA FOR REMOVAL OF INTRACEREBRAL HEMORRHAGE AND OTHER LESIONS

(71) Applicant: THE REGENTS OF THE UNIVERSITY OF CALIFORNIA, Oakland, CA (US)

(72) Inventor: Neil Martin, Encino, CA (US)

(73) Assignee: THE REGENTS OF THE UNIVERSITY OF CALIFORNIA, Oakland, CA (US)

(21) Appl. No.: 15/399,446

(22) Filed: Jan. 5, 2017

Related U.S. Application Data
(63) Continuation of application No. PCT/US2015/039443, filed on Jul. 7, 2015.
(60) Provisional application No. 62/022,010, filed on Jul. 8, 2014.
(60) Provisional application No. 62/021,893, filed on Jul. 8, 2014.

(51) Int. Cl. A61M 1/00; A61B 17/22; A61B 17/34; A61B 17/3207; A61M 3/02; A61B 17/16

(52) U.S. Cl. CPC A61M 1/0058; A61M 3/0283; A61M 1/0031; A61B 17/1695; A61B 17/3421; A61B 17/3207; A61B 17/22; A61M 2210/0693; A61M 2205/3344; A61B 2017/320733; A61B 2017/22079

(57) ABSTRACT

A minimally invasive surgery device for image-guided removal of lesions in the brain by providing suction and irrigation through a small cranial opening. One particular use is for evacuation of intracerebral hemorrhage. Other uses may include evacuation of pus from an abscess, or cystic fluid or necrotic debris from a tumor.

### CROSS-REFERENCE TO RELATED APPLICATIONS

This application is a 35 U.S.C. §111(a) continuation of PCT international application number PCT/US2015/039443 filed on Jul. 7, 2015, incorporated herein by reference in its entirety, which claims priority to, and the benefit of, U.S. provisional patent application Ser. No. 62/021,893 filed on Jul. 8, 2014, incorporated herein by reference in its entirety, and which also claims priority to, and the benefit of, U.S. provisional patent application Ser. No. 62/022,010 filed on Jul. 8, 2014, incorporated herein by reference in its entirety. Priority is claimed to each of the foregoing applications.

The above-referenced PCT international application was published as PCT International Publication No. WO 2016/007553 on Jan. 14, 2016, which publication is incorporated herein by reference in its entirety.

### STATEMENT REGARDING FEDERALLY SPONSORED RESEARCH OR DEVELOPMENT

Not Applicable

### INCORPORATION-BY-REFERENCE OF COMPUTER PROGRAM APPENDIX

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND

1. Technical Field

This description pertains generally to stereotactic evacuation, and more particularly to stereotactic evacuation of intracerebral fluids and/or debris.

2. Background Discussion

Spontaneous intracerebral hemorrhage is a growing health concern whose incidence is 10-30 cases per 100,000 people worldwide. In the US alone, approximately 67,000 people are affected by spontaneous intracerebral hemorrhage each year, and this number is expected to double over the next 40 years as the population continues to age. The lifetime cost of spontaneous intracerebral hemorrhage per patient is estimated to be $123,565.

The failure of open craniotomy to improve the outcomes of patients with spontaneous intracerebral hemorrhage has led to the development of minimally invasive techniques. Studies involving these techniques have shown a trend toward improved outcomes compared with outcomes after medical management.

There is currently no simple cannula for fluid/tissue evacuation of intracerebral hemorrhage.

### BRIEF SUMMARY

Accordingly, the present technology offers an instrument that is specifically configured for image guided or stereotactic evacuation of intracerebral hemorrhage or other lesions. Unique features include simplicity of design; a transparent cannula; a fixation platform for attachment of other instruments, variable diameter and variable length cannulas, and configuration within a re-usable or disposable kit as described. Another unique feature is availability of the rotational clot or tissue fragmentation device.

Further aspects of the technology will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWING(S)

The technology described herein will be more fully understood by reference to the following drawings which are for illustrative purposes only:

FIG. 1 is a schematic diagram of the removal device of the present description.

FIG. 2 shows a schematic side view of a set or kit of cannulas for use with the system of FIG. 1.

FIG. 3 is a close up view of an irrigation channel and the wall of the cannula of FIG. 1.

FIG. 4 is a schematic view of a boring system that may optionally be used for generating an aperture in the patient's skull.

FIG. 5 shows a side view of a tissue disrupting instrument in accordance with the present description.

FIG. 6 shows a side view of the instrument of FIG. 5 installed in a sheath for delivery to a target region of tissue or lumen within the body.

FIG. 7 illustrates a schematic diagram of an exemplary intracerebral hemorrhage/lesion removal procedure using the fluid/debris removal system of FIG. 1.

FIG. 8 illustrates a flow diagram in accordance with the schematic diagram of FIG. 7 for a method of treating a patient with a clot in accordance with the present description.

### DETAILED DESCRIPTION

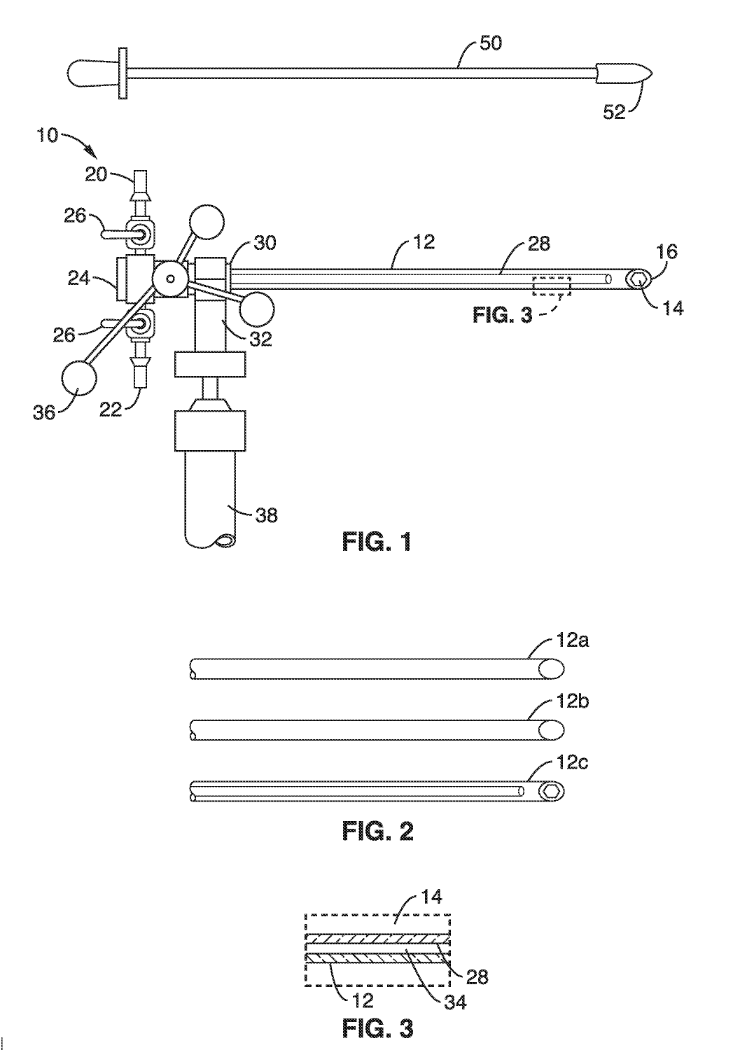

Referring to FIG. 1 through FIG. 8, the technology described herein provides a minimally invasive surgery device for image-guided removal of lesions in the brain by providing suction and irrigation through a burr hole (i.e. small cranial opening). One particular use of the system of the present description is for evacuation of intracerebral hemorrhage. Other uses may include evacuation of pus from an abscess, or cystic fluid or necrotic debris from a tumor. Additional types of lesional removal may be appropriate as well, as are other uses of the device.

As shown in a preferred embodiment in FIG. 1, the fluid/debris removal system 10 comprises a cannula 12 with two suction ports 20 and 22 that are positioned relatively normal to and in fluid communication with a central channel 14 of the cannula. Valves 26 control flow to or from the ports 20, 22 into the cannula channel 14. Port 24 at the proximal end of the cannula 12 provides access for instrumentation contained in sheath 28, an endoscope, or the like. Port 24 may be constructed from a compliant material that stretches to facilitate different sizes of endoscopes, and may also include an end plug (not shown).

In one embodiment, the cannula 12 may include a reinforced segment or housing 30 for attachment of various instruments, e.g. a self-retaining instrument holder (manual or hydraulic), and/or an image-guidance system optical or electromagnetic registration attachment 38 via clamp 32. Housing 30 may be configured to support suction ports 20, and 22 at proximal end, in addition to one or more reflective spheres 36 for motion tracking (e.g. frameless stereotactic tracking). Registration attachment 38 may comprise an end-adapter for fixation of a variety of endoscopes, which would be used to inspect the central lumen 14 of the cannula 12.

The system 10 may also include an obturator 50 having a rounded-distal point 52 sized to occlude channel 14 at distal end 16 of the cannula 12. In various embodiments, the cannula 12 and obturator 50 may be made as a re-usable, sterilizable device, or as a one-time-use disposable device.

Referring to FIG. 2, the system 10 may comprise a set or kit of cannulas comprising different materials and/or sizes. Cannula 12a may be made of steel or other appropriate metal or alloy. Cannula 12b may comprise a non-radio-opaque material configured for use with X-ray, computerized tomography, etc., or may comprise a non-magnetic material for use with MRI image guidance. In a preferred embodiment, the cannula is configured for stereotactic image guidance or interface with frameless stereotactic systems, by inclusion of a registration attachment (e.g. 3-ball star 36 other image registration device for compatibility with existing registration devices).

Cannula 12c may be constructed of glass (for transparency); of plastic, or of other opaque or transparent material. The transparency of cannula 12c may allow for visualization of the surrounding tissue, for instance by an endoscope, through the wall of the cannula 12c.

The system 10 may also include a kit of cannulas 12 with corresponding obturators 50 having of various diameters—e.g., 6 mm, 7 mm, 8 mm, 9 mm (or larger or smaller); and various lengths—e.g., 8 cm, 12 cm, 15 cm, 20 cm (or larger or smaller).

Referring to FIG. 1 and FIG. 3, an irrigation channel 34 may be incorporated into the wall of the cannula 12. A corresponding irrigation port may be provided through any of ports 20, 22 or 24.

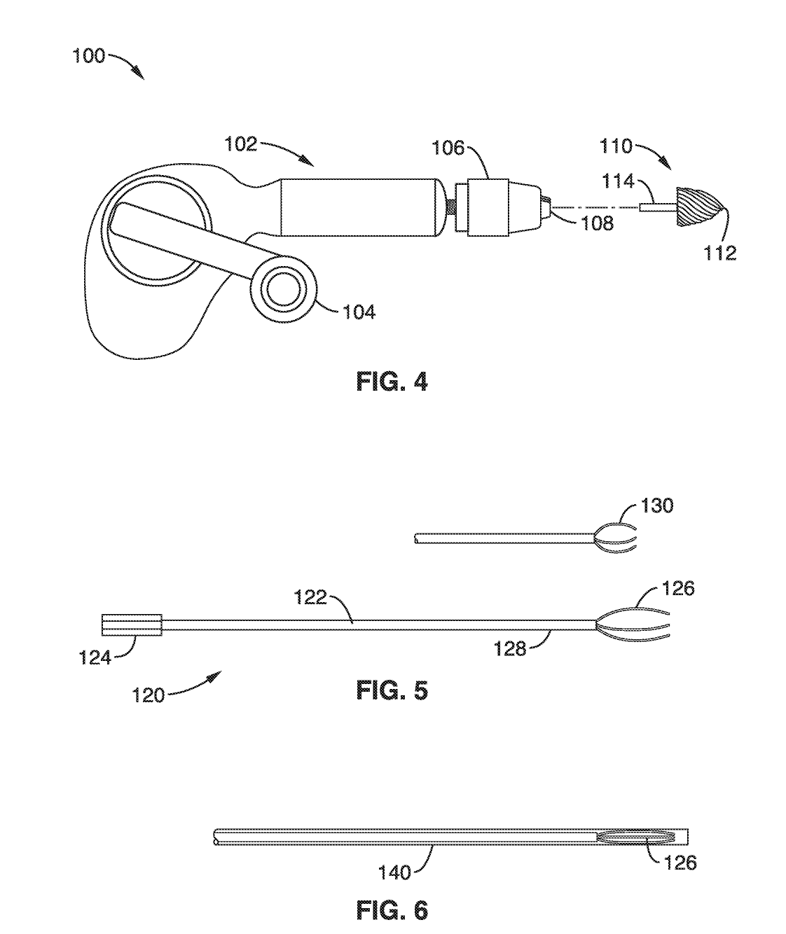

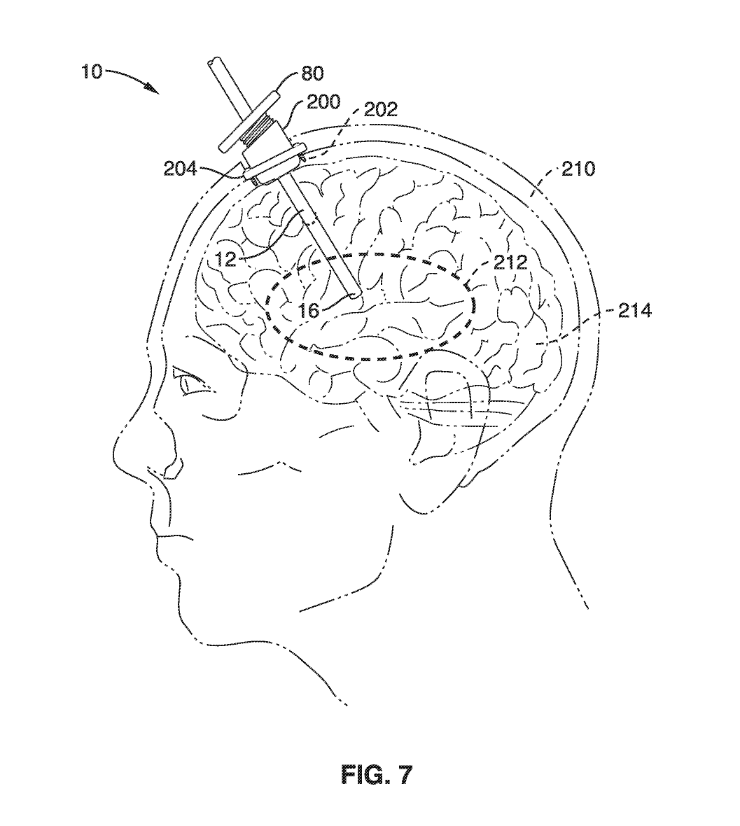

Referring to FIG. 4, a boring system 100 may optionally be used for generating an aperture 204 in the patient's skull 210 (see FIG. 7). System 100 includes a drill 102 and bit 110 having an acorn-shaped burr 112 for generating the appropriate aperture. Extension 114 of bit 110 is configured to be detachably coupled to radial drive portion 106 of drill 102 via locking clamp 108. Rotation of handle 104 affects rotation of drive portion 106 and installed bit 110.

Referring to FIG. 5 and FIG. 6, the system 10 may further include a tissue disrupting instrument 120 having a separately-introduced (e.g. via port 24 and channel 14 in cannula 12) rotatable central shaft 122 with a distal end 128 attached to a bladed array of wires 126 forming an “egg-beater” configuration for fragmentation of a clot or other anatomical formation/feature. Proximal end 124 of shaft 122 is configured for releasable attachment to a rotation device such as drill 100. As seen in FIG. 5, distal end 128 may also comprise a closed-end configuration 130 aside from the open-end configuration 126. FIG. 6 shows a side view of instrument 120 installed in a sheath 140 for delivery through channel 14 and to a target region of tissue or lumen within the body.

FIG. 7 illustrates a schematic diagram of an exemplary intracerebral hemorrhage/lesion removal procedure using the fluid/debris removal system 10 of the present description. The distal end 16 of the cannula 12 is inserted through aperture 204 in skull 210 and into the target treatment region 212 (e.g. hemorrhage, tumor, etc.) within the patient's brain 214. The housing 32 may be compatible with existing commercial components, and comprise a clamp 80 that is compatible with a cranial mounting fixation device (e.g. Medtronic Navigus 200, or the like) that is attached to the skull at aperture 204 via screws 202.

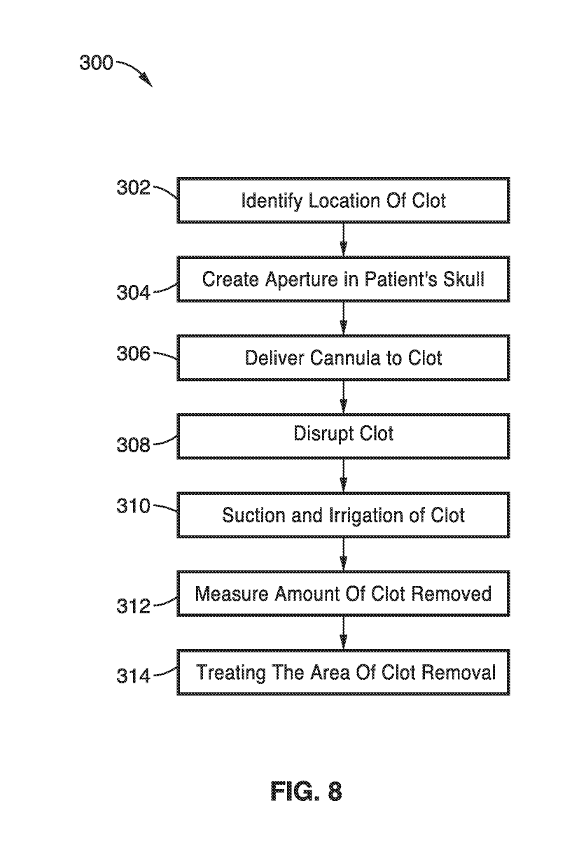

FIG. 8 illustrates a flow diagram in accordance with the schematic diagram of FIG. 7 for a method 300 of treating a patient with a clot in accordance with the present description. The first step 302 comprises identifying the location of a clot. This may be performed via fluoroscopic (or other imaging modality, e.g. CT MRI, etc.) guidance. Next, the drill 100, or similar device is used at step 304 for creating opening or aperture 204 in the patient's skull 210 for access. Next, the cannula 12 is inserted into the clot 212 at step 306. This step may first include installing Medtronic Navigus 200 or similar device. If necessary, the clot may be disrupted at step 308 with instrument 120 to loosen the clot 212 for removal.

At step 310, suction and irrigation is applied via cannula 12 to remove the clot. System 10 may include a regulator (not shown) coupled to one or more of inputs 20 or 22 to adjust suction pressure from “wall suction.”

At step 312, the physician may optionally measure the amount of clot removed for verification, and treating the area of clot removal. In one embodiment, System 10 may include a “trap” (graduated cylinder—not shown) coupled to one or more of inputs 20 or 22 to collect and measure the volume of evacuated fluid, and other material. Removal may also be verified via visual inspection either with an endoscope or external imaging source. If removal is found to be incomplete, step 310 and 312 (and optionally 308) may be repeated.

At step 314, additional treatments may optionally be performed on the target treatment region. Treatments may include infusion of drugs, stem cells, or liquids or gels that promote healing. As an additional optional treatment procedure, an array of several small tubes (not shown) having preformed shapes and varying lengths may be inserted through the sheath 28 (see FIG. 3) and cannula 12 and left in place in the brain at clot 212 after the sheath 28 and cannula 12 are removed, with the tube array having the effect of infusing treatment agents.

In various embodiments, system 10 as detailed above may comprise a kit of one or more of the above components, and configured to be fixated by self-retaining retractors, by burr-hole fitted fixation devices, by manual adjustable tool or endoscope holders, or by fluid/air hydraulic fixation devices. The kit may be reusable, (i.e. non-disposable), or configured to be disposable for one-time use. The kit may be provided with or without a matched endoscope.

The system 10 and components detailed above may be embodied in various additional ways. For example, the system 10 may be fabricated as a re-usable device, or preferably as a single-use disposable device that can be included as part of an entire surgical package. Various components of the system 10 may preferably be manufactured from CT/MRI compatible materials, such as non-ferromagnetic materials and can be made radio-opaque.

From the description herein, it will be appreciated that that the present disclosure encompasses multiple embodiments which include, but are not limited to, the following:

1. An apparatus for intracerebral removal of fluid and/or debris, the apparatus comprising: a cannula; the cannula having a central channel terminating at an opening at a distal end of the cannula; the cannula configured to be delivered through an aperture in a patient's skull for delivery to a treatment region within the cranium of the patient; a suction port located at a proximal location of the cannula, the suction port in fluid communication with the central channel for evacuating fluid and/or debris from the target treatment region; and an irrigation port disposed at a proximal location of the cannula, the irrigation port in fluid communication with the distal end of the cannula for delivering fluid to the target treatment region.

2. The apparatus of any preceding embodiment, wherein the irrigation port is coupled to the distal end of the cannula via an irrigation channel disposed within a wall of the cannula.

3. The apparatus of any preceding embodiment, wherein the cannula includes a reinforced segment at the proximal end of the cannula for attachment of one or more devices.

4. The apparatus of any preceding embodiment, wherein the one or more devices comprises one or more of: a self-retaining instrument holder, an image-guidance system optical and electromagnetic registration attachment.

5. The apparatus of any preceding embodiment, wherein the one or more devices comprises a cranial mounting fixation device.

6. The apparatus of any preceding embodiment, further comprising:

a rounded-point obturator; the obturator configured to be disposed in the central channel to occlude the opening at the distal end of the cannula during delivery of the cannula to the treatment region.

7. The apparatus of any preceding embodiment, further comprising a central port coupled to the central channel, the central port configured for insertion of an endoscope to inspect a central lumen of the cannula and extend through the distal opening of the cannula.

8. The apparatus of any preceding embodiment, further comprising tissue disruption instrument comprising a separately-introduced rotatable central shaft attached to a bladed array of wires for fragmentation of a clot within the treatment region.

9. The apparatus of any preceding embodiment, further comprising fluid trap in fluid communication with the suction port to collect and measure a volume of evacuated fluid and debris from the treatment region.

10. The apparatus of any preceding embodiment, further comprising a suction regulator in fluid communication with the suction port to adjust suction pressure delivered to the treatment region.

11. A method for intracerebral removal of fluid and/or debris, the method comprising: identifying a target treatment location within a patient's brain; creating an opening in the patient's skull; inserting a cannula through the skull and into the target treatment location; irrigating the target treatment location; and applying suction to the target treatment location via distal end of the cannula to remove fluid and/or debris from the target treatment location.

12. The method of any preceding embodiment, further comprising coupling the cannula to a cranial mounting fixation device prior to inserting the cannula.

13. The method of any preceding embodiment, wherein the target treatment location comprises a clot, and wherein the clot is disrupted with an instrument to loosen debris from the clot.

14. The method of any preceding embodiment, further comprising:

measuring the amount of clot removed.

15. The method of any preceding embodiment, wherein measuring the amount of clot removed comprises trapping evacuated debris and/or fluids within a container to measure a volume of the removed debris and/or fluids.

16. The method of any preceding embodiment, further comprising:

treating the location of clot removal.

17. The method of any preceding embodiment, wherein treating the location comprises infusion of drugs, or stem cells, or liquids or gels to promote healing.

18. The method of any preceding embodiment, wherein treating the location comprises delivering an array of small tubes having preformed shapes and varying lengths through the cannula and into the location, the array of small tubes configured for promoting infusion of one or more treatment agents.

19. A system for intracerebral removal of fluid and/or debris, the apparatus comprising: a kit of cannulas; wherein a cannula is configured to be selected from the kit of cannulas based on one or more of a cannula dimension and material selection; the cannula having a central channel terminating at an opening at a distal end of the cannula; the cannula configured to be delivered through an aperture in a patient's skull for delivery to a treatment region within the cranium of the patient; a suction port located at a proximal location of the cannula, the suction port in fluid communication with the central channel for evacuating fluid and or debris from the target treatment region; and an irrigation port disposed at a proximal location of the cannula, the irrigation port in fluid communication with the distal end of the cannula for delivering fluid to the target treatment region.

20. The system of any preceding embodiment, wherein the irrigation port is coupled to the distal end of the cannula via an irrigation channel disposed within a wall of the cannula.

21. The system of any preceding embodiment, wherein the cannula includes a reinforced segment at the proximal end of the cannula for attachment of one or more devices.

22. The system of any preceding embodiment, wherein the one or more devices comprises one or more of: a self-retaining instrument holder, an image-guidance system optical and electromagnetic registration attachment.

23. The system of any preceding embodiment, wherein the one or more devices comprises a cranial mounting fixation device.

24. The system of any preceding embodiment, further comprising: a rounded-point obturator; the obturator configured to be disposed in the central channel to occlude the opening at the distal end of the cannula during delivery of the cannula to the treatment region.

25. The system of any preceding embodiment, further comprising a central port coupled to the central channel, the central port configured for insertion of an endoscope to inspect a central lumen of the cannula and extend through the distal opening of the cannula.

26. The system of any preceding embodiment, further comprising tissue disruption instrument comprising a separately-introduced rotatable central shaft attached to a bladed array of wires for fragmentation of a clot within the treatment region.

27. The system of any preceding embodiment, further comprising fluid trap in fluid communication with the suction port to collect and measure a volume of evacuated fluid and debris from the treatment region.

28. The system of any preceding embodiment, further comprising a suction regulator in fluid communication with the suction port to adjust suction pressure delivered to the treatment region.

29. The system of any preceding embodiment, further comprising: a drill configured for generating the aperture in the patient's skull.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. An apparatus for intracerebral removal of fluid and/or debris, the apparatus comprising:
a cannula;
the cannula having a central channel terminating at an opening at a distal end of the cannula;
the cannula configured to be delivered through an aperture in a patient's skull for delivery to a treatment region within the cranium of the patient;
a suction port located at a proximal location of the cannula, the suction port in fluid communication with the central channel for evacuating fluid and/or debris from the target treatment region; and
an irrigation port disposed at a proximal location of the cannula, the irrigation port in fluid communication with the distal end of the cannula for delivering fluid to the target treatment region.

2. The apparatus of claim 1, wherein the irrigation port is coupled to the distal end of the cannula via an irrigation channel disposed within a wall of the cannula.

3. The apparatus of claim 1, wherein the cannula includes a reinforced segment at the proximal end of the cannula for attachment of one or more devices.

4. The apparatus of claim 3, wherein the one or more devices comprises one or more of: a self-retaining instrument holder, an image-guidance system optical and electromagnetic registration attachment.

5. The apparatus of claim 3, wherein the one or more devices comprises a cranial mounting fixation device.

6. The apparatus of claim 1, further comprising:
a rounded-point obturator;
the obturator configured to be disposed in the central channel to occlude the opening at the distal end of the cannula during delivery of the cannula to the treatment region.

7. The apparatus of claim 1, further comprising a central port coupled to the central channel, the central port configured for insertion of an endoscope to inspect a central lumen of the cannula and extend through the distal opening of the cannula.

8. The apparatus of claim 1, further comprising tissue disruption instrument comprising a separately-introduced rotatable central shaft attached to a bladed array of wires for fragmentation of a clot within the treatment region.

9. The apparatus of claim 1, further comprising fluid trap in fluid communication with the suction port to collect and measure a volume of evacuated fluid and debris from the treatment region.

10. The apparatus of claim 1, further comprising a suction regulator in fluid communication with the suction port to adjust suction pressure delivered to the treatment region.

11. A method for intracerebral removal of fluid and/or debris, the method comprising:
identifying a target treatment location within a patient's brain;
creating an opening in the patient's skull;
inserting a cannula through the skull and into the target treatment location;
irrigating the target treatment location; and
applying suction to the target treatment location via distal end of the cannula to remove fluid and/or debris from the target treatment location.

12. A method as recited in claim 11, further comprising coupling the cannula to a cranial mounting fixation device prior to inserting the cannula.

13. A method as recited in claim 11, wherein the target treatment location comprises a clot, and wherein the clot is disrupted with an instrument to loosen debris from the clot.

14. A method as recited in claim 13, further comprising: measuring the amount of clot removed.

15. A method as recited in claim 14, wherein measuring the amount of clot removed comprises trapping evacuated debris and/or fluids within a container to measure a volume of the removed debris and/or fluids.

16. A method as recited in claim 13, further comprising:
treating the location of clot removal.

17. A method as recited in claim 16, wherein treating the location comprises infusion of drugs, or stem cells, or liquids or gels to promote healing.

18. A method as recited in claim 16, wherein treating the location comprises delivering an array of small tubes having preformed shapes and varying lengths through the cannula and into the location, the array of small tubes configured for promoting infusion of one or more treatment agents.

19. A system for intracerebral removal of fluid and/or debris, the apparatus comprising:
a kit of cannulas;
wherein a cannula is configured to be selected from the kit of cannulas based on one or more of a cannula dimension and material selection;
the cannula having a central channel terminating at an opening at a distal end of the cannula;
the cannula configured to be delivered through an aperture in a patient's skull for delivery to a treatment region within the cranium of the patient;
a suction port located at a proximal location of the cannula, the suction port in fluid communication with the central channel for evacuating fluid and or debris from the target treatment region; and
an irrigation port disposed at a proximal location of the cannula, the irrigation port in fluid communication with the distal end of the cannula for delivering fluid to the target treatment region.

20. The system of claim 19, wherein the irrigation port is coupled to the distal end of the cannula via an irrigation channel disposed within a wall of the cannula.

21. The system of claim 19, wherein the cannula includes a reinforced segment at the proximal end of the cannula for attachment of one or more devices.

22. The system of claim 21, wherein the one or more devices comprises one or more of: a self-retaining instrument holder, an image-guidance system optical and electromagnetic registration attachment.

23. The system of claim 21, wherein the one or more devices comprises a cranial mounting fixation device.

24. The system of claim 19, further comprising:
a rounded-point obturator;
the obturator configured to be disposed in the central channel to occlude the opening at the distal end of the cannula during delivery of the cannula to the treatment region.

25. The system of claim 19, further comprising a central port coupled to the central channel, the central port configured for insertion of an endoscope to inspect a central lumen of the cannula and extend through the distal opening of the cannula.

26. The system of claim 19, further comprising tissue disruption instrument comprising a separately-introduced rotatable central shaft attached to a bladed array of wires for fragmentation of a clot within the treatment region.

27. The system of claim 19, further comprising fluid trap in fluid communication with the suction port to collect and measure a volume of evacuated fluid and debris from the treatment region.

28. The system of claim 19, further comprising a suction regulator in fluid communication with the suction port to adjust suction pressure delivered to the treatment region.

29. The system of claim 19, further comprising:
a drill configured for generating the aperture in the patient's skull.
